(19) United States
(12) Patent Application Publication
(10) Pub. No.: US 2004/0126331 A1
(43) Pub. Date: Jul. 1, 2004

(54) Use of an oral composition

(75) Inventors: Ruth Ann Corcoran, Surbiton (GB); Trevor Neil Day, Egham (GB); Mark Ieuan Edwards, Horsell Woking (GB); Sheri Anne Hunt, Memphis, TN (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/675,393

(22) Filed: Sep. 30, 2003

Related U.S. Application Data
(60) Provisional application No. 60/416,069, filed on Oct. 4, 2002.

(51) Int. Cl. A61K 7/16

(52) U.S. Cl. 424/49

(57) ABSTRACT

Liquid dentifrice compositions are provided which give improved cleaning performance and flavour display when used in the small amounts that can be dispensed onto a typical electric toothbrush head. The liquid dentifrice has a viscosity of less than about 500 Pa.s at 0.1 s⁻¹ at 25° C. Representative compositions include greater than about 2% by weight of a surfactant; greater than about 1.2% flavour and greater than 50% liquid carrier materials, including controlled amounts of water and humectants, or alternatively polyoxyethylene thickeners.

### CROSS REFERENCE TO RELATED APPLICATION

This application claims priority under 35 USC 119(e) to U.S. Provisional Application 60/416,069, filed Oct. 4, 2002.

### FIELD OF THE INVENTION

The present invention relates to a liquid dentifrice composition with improved flavour and foaming, suitable for use in small amounts with an electric toothbrush.

### BACKGROUND OF THE INVENTION

It remains the case that most regular oral treatments are provided via the application of a single dentifrice product to a toothbrush which is manually rubbed against the teeth. Within the last two years there has been a substantial increase in the market penetration of electric toothbrushes which, historically rather expensive, have become much more affordable and therefore more widespread. The basic process however, of squeezing toothpaste onto the brush head remains the same.

Whilst electric toothbrushes have the capability to improve brushing performance, and much effort has been put against their design, very little attention has been paid to optimizing dentifrice products for use with electric toothbrushes. A number of problems arise with using regular dentifrice products with electric toothbrushes. Firstly, users are used to dosing their dentifrice by dispensing it in a ribbon along the toothbrush head. Electric toothbrushes however often have relatively small heads which are physically incapable of holding a typical dose of paste as it would be applied to a manual toothbrush of typical construction. This can lead to under dosing of product with consequently reduced efficacy. Secondly, a typical electric toothbrush head vibrates or oscillates very rapidly. With thick, pasty products, particularly with stiff gels, this can lead either to product being thrown off the brush head if the user inadvertently switches it on before putting the brush head in his mouth, or to the product not being applied where intended even if it successfully reaches the mouth. A further problem arises from the vibrations of the electric brush. An electric brush can also stimulate increased saliva production by the user. This has the effect of further reducing product concentration above and beyond that arising, as above, from lower absolute dosage, further reducing cleaning performance. A particular problem arising from the smaller amounts used is the reduction in foaming capability. Although foaming can be partially boosted by an increase in foaming surfactant and partially compensated for by the use of polymeric foam stabilisers, the use of the latter can lead to unacceptable thickening. It has now surprisingly been found that the use of a high molecular weight polyoxyethylene surfactant can improve foaming performance without unacceptable viscosity build.

European patent application EP 867 173 discloses two part dentifrice formulations which comprise polyoxyethylene but does not disclose its use as a foam stabiliser in liquid dentifrice compositions.

U.S. Pat. No. 4,383,987 also discloses dentifrice formulations which comprise polyoxyethylene but always in combination with considerable amounts of other thickeners.

It is an object of the present invention to provide a liquid dentifrice which at least partially settles into the brush bristles shortly after dispensing, to address the problem of physical loss from the brush head.

It is a further object of the present invention to provide a liquid dentifrice with improved foaming when used in small amounts, in particular with an electric toothbrush.

Liquid dentifrice products are well known in the patent literature. Mention is made, for example, of U.S. Pat. No. 5,695,746 which deals with increased menthol impact from a liquid dentifrice; WO 95/22958 dealing with improved transparency from a liquid dentifrice with an abrasive; and WO 00/19970 relating to transparent liquid dentifrice with an improved rheology for dispensing from flexible bottles.

It is a further object of the present invention to provide a liquid dentifrice with improved flavour and cleaning when used in small amounts, in particular with an electric toothbrush. It is yet a further object of the present invention to provide a liquid dentifrice which can be pumped from a product reservoir integrated within the brush.

These and other objects of the present invention will become more readily apparent from consideration of the following summary, detailed description and examples.

### SUMMARY OF THE INVENTION

A liquid dentifrice composition is provided which includes greater than about 2% by weight of a surfactant; greater than about 1.2% flavour and greater than 50% liquid carrier materials comprising water and humectants in a ratio of from 0.65 to 1.5, the liquid dentifrice having a viscosity of less than about 500 Pa.s at 0.1 s-1 at 25° C.

The dentifrice provides improved cleaning performance and flavour display when used in the small amounts that can be dispensed onto a typical electric toothbrush head.

A further aspect of the invention relates to a liquid dentifrice having a viscosity of less than 500 Pa.s at 0.1 s⁻¹ at 25° C. and comprising:

a) at least 0.1% of a polyoxethylene having a molecular weight greater than 200,000;

b) at least 2% of a dental abrasive; and

c) greater than 50% liquid carrier materials.

The invention further relates to a method of cleaning teeth with a small amount of the dentifrice and to a toothbrush with an integrated reservoir containing the dentifrice, the toothbrush being adapted to dispense a small unit dose.

In yet a further aspect, the invention relates to the liquid dentifrices, wherein the liquid dentifrice is contained within a package which has a dispensing orifice of cross-sectional area of less than about 10 mm2. The packaged dentifrice is optionally combined with a set of instructions for dispensing onto and use with an electric toothbrush. Alternately, the packaged dentifrice can be a replaceable reservoir for use with an electric toothbrush having an integrated dentifrice reservoir and the optional instructions may comprise direction for replacing the reservoir in the toothbrush.

All parts, percentages and proportions referred to herein and in the appended claims are by weight of the total dentifrice composition unless otherwise indicated. All measurements are made at 25° C. on the total dentifrice composition unless otherwise indicated.

### DETAILED DESCRIPTION OF THE INVENTION

The dentifrice of this invention is a liquid composition having a viscosity of less than about 500 Pa.s at 0.1 s⁻¹ at 25° C. The dentifrice can be a single composition or can be supplied in two or more parts provided that the parts are intended to be mixed together prior to or during use. If the dentifrice is supplied as a two or more part composition then weight percentages and viscosity measurements relate to the total combined composition, mixing as necessary to achieve uniformity and performing viscosity measurements immediately after obtaining a uniform composition. Each individual part should also meet the viscosity criterion herein. By “liquid” is meant that the product will, under its own weight, at least partially flow between the bristles of a tufted toothbrush head, having a typical configuration of tuft diameters of about 1-1.5 mm and spacing between tufts of similar size, within a time frame of 30 seconds. Viscosity can conveniently be measured using the Carrimed CSL 100 Controlled Stress Rheometer with a 4 cm diameter parallel plate measuring system and a 1000 micron gap between the plates. Preferably the viscosity is less than about 400 Pa.s, more preferably less than about 300 Pa.s at 0.1 s⁻¹ at 25° C. In preferred embodiments the viscosity is at least about 5 Pa.s and more preferably at least about 15 Pa.s at 0.1 s⁻¹ at 25° C.

Additionally, it is preferable that the liquid dentifrice of the present invention, when diluted to a 16.67% slurry in water has a viscosity of greater than 0.1 Pa.s, preferably greater than 0.2 Pa.s, more preferably greater than 0.3 Pa.s at a shear rate of 1 s⁻¹. It has been found that liquid dentifrices with dilution viscosities greater than 0.1 Pa.s at a shear rate of 1 s⁻¹ induce greater consumer compliance and better cleaning efficiency. In particular it has been found to provide a foam viscosity high enough to enable its retention in the oral cavity without excessive manipulation or pressure application of the brush by the consumer. Reduction of the exerted pressure results in a better and more efficient cleaning action, and decreased irritation of the oral tissues. More particularly, and without wishing to be bound by theory, it is believed that the foam viscosity thus provided is particularly beneficial when used in conjunction with a sonic toothbrush, such as that marketed under the trade name Sonicare®, since the cleaning action of such brushes depends at least in part on the transmission of pressure waves through the foam or the dentifrice diluted by saliva, the pressure wave effect being damped by pressure of the brush against the teeth. By “sonic toothbrush” is meant a brush whose bristle tips move at a velocity greater than 1.25 meters per second, typically having a head oscillating in the frequency range of from 200 to 500 Hz. The polyoxyethylene described herein has been found to be beneficial in providing the required diluted viscosity but other thickeners such as polysaccharides at a level of 0.6% or greater can also be valuable.

Surfactants

The liquid dentifrice of the present invention preferably comprises greater than about 2% by weight of a surfactant or mixture of surfactants, though lower levels, such as 1%, may be employed when the polyoxyethylene surfactant, or another foam stabiliser, is used. Surfactant levels cited herein are on a 100% active basis, even though common raw materials such as sodium lauryl sulphate may be supplied as aqueous solutions of lower activity.

The surfactant is important for oral cleaning, both through removal of dirt from surfaces and in foam generation to suspend removed dirt. Suitable surfactant levels are from about 2% to about 15%, preferably from about 2.2% to about 10%, more preferably from about 2.5 to about 5% by weight of the total composition. Suitable surfactants for use herein include anionic, amphoteric, non-ionic, zwitterionic and cationic surfactants, though anionic, amphoteric, non-ionic and zwitterionic surfactants (and mixtures thereof) are preferred.

Preferred for use herein are anionic surfactants. Useful anionic surfactants herein include the water-soluble salts of alkyl sulphates and alkyl ether sulphates having from 10 to 18 carbon atoms in the alkyl radical and the water-soluble salts of sulphonated monoglycerides of fatty acids having from 10 to 18 carbon atoms. Sodium lauryl sulphate and sodium coconut monoglyceride sulphonates are examples of anionic surfactants of this type. Sodium lauryl sulphate is preferred. In preferred embodiments, the dentifrice herein comprises at least about 0.5%, preferably at least about 1.5%, more preferably at least about 2% anionic surfactant.

Other useful surfactants include alkali metal or ammonium salts of sarcosinates, isethionates and taurates. Preferred herein are the sodium and potassium salts of the following: lauroyl sarcosinate, myristoyl sarcosinate, palmitoyl sarcosinate, stearoyl sarcosinate and oleoyl sarcosinate.

Other suitable compatible surfactants, which can optionally be used in combination with the surfactants above include those mentioned in U.S. Pat. No. 3,959,458; U.S. Pat. No. 3,937,807; and U.S. Pat. No. 4,051,234.

Preferred cationic surfactants useful in the present invention can be broadly defined as derivatives of aliphatic quaternary ammonium compounds having one long alkyl chain containing from about 8 to 18 carbon atoms such as lauryl trimethylammonium chloride; cetyl pyridinium chloride; benzalkonium chloride; cetyl trimethylammonium bromide; di-isobutylphenoxyethyl-dimethylbenzylammonium chloride; coconut alkyltrimethylammonium nitrite; cetyl pyridinium fluoride; etc. Preferred compounds are the quaternary ammonium fluorides described in U.S. Pat. No. 3,535,421, where said quaternary ammonium fluorides have detergent properties. Certain cationic surfactants can also act as germicides in the compositions disclosed herein. Some cationic agents such as chlorhexidine, although suitable for use in the current invention, are not preferred due to their capacity to stain the oral cavity's hard tissues. Persons skilled in the art are aware of this possibility and should incorporate cationics only with this limitation in mind.

Preferred nonionic surfactants that can be used in the compositions of the present invention can be broadly defined as compounds produced by the condensation of alkylene oxide groups (hydrophilic in nature) with an organic hydrophobic compound which may be aliphatic and/or aromatic in nature. Examples of suitable nonionic surfactants include the poloxamers; sorbitan derivatives, such as sorbitan di-isostearate; ethylene oxide condensates of hydrogenated castor oil, such as PEG-30 hydrogenated castor oil; ethylene oxide condensates of aliphatic alcohols or alkyl phenols; products derived from the condensation of ethylene oxide with the reaction product of propylene oxide and ethylene diamine; long chain tertiary amine oxides; long chain tertiary phosphine oxides; long chain dialkyl sulphoxides and mixtures of such materials.

Particularly preferred for use herein are polyoxyethylene surfactants having a molecular weight greater than about 200,000. These materials are useful for stabilising foams without contributing to excess viscosity build for the dentifrice. These materials are useful for stabilising foams without contributing to excess viscosity build for the dentifrice. Polyoxyethylene is also commonly known as polyethylene glycol (“PEG”) or polyethylene oxide. The polyoxyethylene herein may comprise small amounts of monomers other than ethylene oxide, but no more than about 20%, preferably no more than about 5% by weight of the polyoxyethylene. Preferably, the polyoxyethylene consists entirely of ethylene oxide monomers. The polyoxyethylenes suitable for this invention will have a molecular weight of from about 200,000 to about 7,000,000 or greater. Preferably, the molecular weights will be from 600,000 to 2,000,000, and more preferably from 800,000 to 1,000,000. “Polyox” is the tradename for the high molecular weight polyoxyethylene produced by Union Carbide. A particularly preferred material is known by the INCI name PEG-20M and available from Union Carbide as Polyox WS1105. Useful levels of polyoxyethylene are from about 0.1% to about 20%, preferably from about 0.5% to about 10%, more preferably from about 1% to about 5%.

Zwitterionic surfactants can be broadly described as derivatives of aliphatic quaternary ammonium, phosphonium, and sulphonium compounds, in which the aliphatic radicals can be straight chain or branched, and wherein one of the aliphatic substituents contains from about 8 to 18 carbon atoms and one contains an anionic water-solubilising group, e.g., carboxy, sulphonate, sulphate, phosphate or phosphonate. Preferred zwitterionic surfactants include the betaine surfactants disclosed in U.S. Pat. No. 5,180,577. Typical alkyl dimethyl betaines include decyl betaine or 2-(N-decyl-N,N-dimethylammonio) acetate, coco betaine, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, lauramidopropyl betaine and the like. The betaines of choice are cocoamidopropyl betaine and lauramido propyl betaine.

Flavour

The liquid dentifrice of the present invention preferably comprises greater than about 1.2% flavour, generally added as a flavour oil. Preferred flavour levels in the present liquid dentifrice compositions are from about 1.2% to about 5.0%, more preferably from about 1.4% to about 4.0%, and most preferably from about 1.5% to about 3.0%, by weight of the liquid dentifrice. Typically a flavour oil will be manufactured in a separate step and will comprise multiple components in order to provide a balanced flavour which is acceptable to a broad range of people. The individual components of the flavour oil may be in the form of an oil, liquid, semi-solid, solid, or powder and may be of a natural and/or synthetic flavour origin. The term “flavour” according to the present invention means any essence, either natural or synthetic, or active agent (such as coolant agents), included in a composition to provide a palatable taste profile, excluding sweeteners and diluents such as ethanol or propylene glycol. Various flavours can be used in the present invention. The flavour generally consists of flavour components from the group consisting of peppermint, spearmint, cinnamon, spice, wintergreen, fruit, citrus, herbal, medicinal, and common food flavours (e.g. chocolate) and mixtures thereof. Illustrative, but non-limiting examples of such components include peppermint oils such as Mentha piperita and Mentha arvensis; spearmint oils such as Mentha cardiaca and Mentha spicata; hydrocarbons such as limonene, caryophyllene, myrcene, and humulene; alcohols such as menthol, linalool, 3-decanol, and pinocarveol; ketones such as piperitone, menthone, spicatone, and 1-carvone; aldehydes such as acetaldehyde, 3-hexanal, or n-octanal; oxides such as menthofuran, piperitone oxide, or carvyl acetate-7,7 oxide; acids such as acetic and ocenoic; and sulphides such as dimethyl sulphide. Components also include esters such as menthyl acetate, benzyl isobutyrate, and 3-octyl acetate.

The components may also consist of essential oils such sage oil, parsley oil, marjoram oil, cassia oil, clove bud oil, cinnamon oil, eucalyptus oil, anise oil, and mixtures thereof. The flavour components may also consist of flavour chemicals such as cinnamic aldehyde, eugenol, ionone, anethole, eucalyptol, methyl salicylate, oxanone, alpha-irisone, and mixtures thereof. Preferred are peppermint oils, spearmint oils, menthol, anethole, methyl salicylate, cinnamon oils, clove bud oils, oxanone, and mixtures thereof. Flavour components are described in more detail in Fenaroli's Handbook of Flavor Ingredients, Third Edition, Volumes 1 & 2, CRC Press, Inc. (1995), and Steffen Arctander's Perfume and Flavour Chemicals, Volumes 1 & 2, (1969). The flavour oil may additionally comprise components such as vanillin, ethyl vanillin, heliotropine, propenyl guaethol, vanilla extracts, veratraldehyde, 4-cis-heptenal, diacetyl, butyl lactate, ethyl lactate, methyl-para-tert-butyl phenyl acetate, gamma and delta hexalactone and heptalactone, benzodihydropyrone, butter starter distillate, delta tetradecalactone, butyraldehyde, and mixtures thereof. A physiological cooling agent may also be incorporated into the flavour oil. The coolant can be any of a wide variety of materials. Included among such materials are carboxamides, menthol, acetals, ketals, diols, and mixtures thereof. Preferred coolants in the present compositions are the paramenthane carboxyamide agents such as N-ethyl-p-menthan-3-carboxamide, (known commercially as “WS-3”) and mixtures thereof and menthone glycerine acetal (known commercially as “MGA”). Further disclosure of coolants suitable for the present invention are discussed in WO97/06695, incorporated by reference herein. In order to achieve a balanced flavour, individual flavour components are generally limited to no more than 70% by weight of the total flavour. In particular, menthol can create an unduly bitter impression at high levels and the total menthol level is preferably kept to below about 60%, more preferably below about 55% by weight of the total flavour. By “total menthol” is meant menthol added as a discrete flavour component as well as any menthol amounts delivered from additional flavour components such as spearmint and, especially, peppermint.

Liquid Carrier Materials

The liquid dentifrice of this invention comprises greater than about 50% liquid carrier materials. Water is usually present. Water employed in the preparation of commercially suitable dentifrice should preferably be deionised and free of organic impurities. Water generally comprises from about 10% to 50% by weight of the liquid dentifrice compositions herein. Preferably the compositions include at least about 30% water, suitably from about 30% to about 50% water. These amounts of water include the free water which is added plus that which is introduced with other materials such as with sorbitol and with surfactant solutions.

Generally the liquid carrier will further include one or more humectants. Suitable humectants are water-miscible or materials having a solubility in water of greater than 10 weight percent selected from sugars, sugar alcohols, and other edible polyhydric alcohols, such as polyethylene glycols, having a molecular weight of less than 50,000. Suitable non-limiting examples of polyhydric alcohols include glycerin, sorbitol and xylitol. Suitable non-limiting examples of polyethylene glycols, referred to by their INCI nomenclature, are PEG-6, PEG-9 and PEG-12. Suitable humectant levels are from about 15% to about 60%, preferably from about 30% to about 55%. To provide the best balance of foaming properties and resistance to drying out, the ratio of total water to total humectant is preferably from about 0.65:1 to 1.5:1, preferably from about 0.85:1 to 1.3:1.

Ethanol may also be present in the liquid dentifrice compositions. These amounts may range from 0.5 to 5%, optimally from 1.5 to 3.5% by weight. Ethanol can be a useful solvent and can also serve to enhance the impact of a flavour, though in this latter respect only low levels are usually employed.

Other components

The liquid dentifrice herein will typically comprise a variety of other components such as abrasives, fluoride ion sources, chelating agents, antimicrobials, thickeners, silicone oils and other adjuvants such as preservatives and colouring agents.

Abrasives

A preferred liquid dentifrice component for use herein is a dental abrasive. Abrasives serve to polish the teeth and/or remove surface deposits. The abrasive material contemplated for use herein can be any material which does not excessively abrade dentine. Suitable abrasives include insoluble phosphate polishing agents, include various calcium phosphates such as, for example, dicalcium phosphate, tricalcium phosphate, calcium pyrophosphate, beta-phase calcium pyrophosphate, dicalcium phosphate dihydrate, anhydrous calcium phosphate, insoluble sodium metaphosphate, and the like. Also suitable are chalk-type abrasives such as calcium and magnesium carbonates, silicas including xerogels, hydrogels, aerogels and precipitates, alumina and hydrates thereof such as alpha alumina trihydrate, aluminosilicates such as calcined aluminium silicate and aluminium silicate, magnesium and zirconium silicates such as magnesium trisilicate and thermosetting polymerised resins such as particulate condensation products of urea and formaldehyde, polymethylmethacrylate, powdered polyethylene and others such as disclosed in U.S. Pat. No. 3,070,510, Dec. 25, 1962. Mixtures of abrasives can also be used. The abrasive polishing materials generally have an average particle size of from about 0.1 to about 30 microns, preferably from about 5 to 15 microns.

Silica dental abrasives of various types offer exceptional dental cleaning and polishing performance without unduly abrading tooth enamel or dentin. The silica abrasive can be precipitated silica or silica gels such as the silica xerogels described in Pader et al., U.S. Pat. No. 3,538,230, issued Mar. 2, 1970 and DiGiulio, U.S. Pat. No. 3,862,307, Jun. 21, 1975, for example silica xerogels marketed under the tradename “Syloid” by W. R. Grace & Company, Davison Chemical Division. Suitable precipitated silica materials include those marketed by the J. M.

Huber Corporation under the tradename, “Zeodent®”, particularly the silicas carrying the designation Zeodent® 119 or Zeodent® 118. These silica abrasives are described in U.S. Pat. No. 4,340,583, Jul. 29, 1982 and WO 96/09809, incorporated herein by reference.

Suitable abrasive levels are from about 1% to about 40%, preferably at least about 2%, such as from about 2% to about 20%, more preferably at least about 5%, such as from about 5% to about 15%.

Fluoride ion sources

For anticaries protection, a source of fluoride ion will normally be present in the liquid dentifrice. Fluoride sources include sodium fluoride, potassium fluoride, calcium fluoride, stannous fluoride, stannous monofluorophosphate and sodium monofluorophosphate. Preferred is sodium fluoride. Suitable levels provide from 25 to 2500 ppm of available fluoride ion by weight of the liquid dentifrice.

Chelating agents

Another preferred optional agent is a chelating agent, of value as an anticalculus agent. Suitable chelating agents include organic acids and their salts, such as tartaric acid and pharmaceutically-acceptable salts thereof, citric acid and alkali metal citrates and mixtures thereof. Chelating agents are able to complex calcium found in the cell walls of the bacteria. Chelating agents can also disrupt plaque by removing calcium from the calcium bridges which help hold this biomass intact. However, it is possible to use a chelating agent which has an affinity for calcium that is too high. This results in tooth demineralisation and is contrary to the objects and intentions of the present invention. Preferred chelating agents have a calcium binding constant of about 101 to 10⁵ to provide improved cleaning with reduced plaque and calculus formation. Sodium and potassium citrate are the preferred alkali metal citrates, with sodium citrate being the most preferred. Also preferred is a citric acid/alkali metal citrate combination. Preferred herein are alkali metal salts of tartaric acid. Most preferred for use herein are disodium tartrate, dipotassium tartrate, sodium potassium tartrate, sodium hydrogen tartrate and potassium hydrogen tartrate. The amounts of chelating agent suitable for use in the present invention are about 0.1% to about 2.5%, preferably from about 0.5% to about 2.5% and more preferably from about 1.0% to about 2.5%. The tartaric acid salt chelating agent can be used alone or in combination with other optional chelating agents.

Another group of agents particularly suitable for use as chelating agents in the present invention are the soluble polyphosphates, polyphosphonates, and pyrophosphates which are useful as anticalculus agents. The pyrophosphate salts used in the present compositions can be any of the alkali metal pyrophosphate salts. Specific salts include tetra alkali metal pyrophosphate, dialkali metal diacid pyrophosphate, trialkali metal monoacid pyrophosphate and mixtures thereof, wherein the alkali metals are preferably sodium or potassium. The salts are useful in both their hydrated and unhydrated forms. An effective amount of pyrophosphate salt useful in the present composition is generally enough to provide at least 1.0% pyrophosphate ion, preferably from about 1.5% to about 6%, more preferably from about 3.5% to about 6% of such ions. It is to be appreciated that the level of pyrophosphate ions is that capable of being provided to the composition (i.e., the theoretical amount at an appropriate pH) and that pyrophosphate forms other than P₂O₇⁻⁴ (e.g., (HP₂O₇⁻³)) may be present when a final product pH is established. The pyrophosphate salts are described in more detail in Kirk & Othmer, Encyclopedia of Chemical Technology, Second Edition, Volume 15, Interscience Publishers (1968).

Also useful are the soluble polyphosphates such as sodium tripolyphosphate and sodium hexametaphosphate. Other long chain anticalculus agents of this type are described in WO 98/22079, incorporated herein by reference. Particularly preferred for use herein are sodium polyphosphate salts containing about 15 to about 25 phosphate units.

Still another possible group of chelating agents suitable for use in the present invention are the anionic polymeric polycarboxylates. Such materials are well known in the art, being employed in the form of their free acids or partially or preferably fully neutralised water soluble alkali metal (e.g. potassium and preferably sodium) or ammonium salts. Preferred are 1:4 to 4:1 copolymers of maleic anhydride or acid with another polymerisable ethylenically unsaturated monomer, preferably methyl vinyl ether (methoxyethylene) having a molecular weight (MW) of about 30,000 to about 1,000,000. These copolymers are available for example as Gantrez AN 139 (MW 500,000), AN 119 (MW 250,000) and preferably S-97 Pharmaceutical Grade (MW 70,000), of GAF Chemicals Corporation. Other operative polymeric polycarboxylates include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, MW 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Additional polymeric polycarboxylates are disclosed in U.S. Pat. No. 4,138,477 to Gaffar and U.S. Pat. No. 4,183,914 to Gaffar et al., and include copolymers of maleic anhydride with styrene, isobutylene or ethyl vinyl ether, polyacrylic, polyitaconic and polymaleic acids, and sulphoacrylic oligomers of MW as low as 1,000 available as Uniroyal ND-2.

Antimicrobials

Also useful for inclusion in the compositions of the present invention are antimicrobial agents. A wide variety of antimicrobial agents can be used, including stannous salts such as stannous pyrophosphate and stannous gluconate; zinc salt, such as zinc lactate and zinc citrate; copper salts, such as copper bisglycinate; quaternary ammonium salts, such as cetyl pyridinium chloride and tetradecylethyl pyridinium chloride; bis-biguanide salts; and nonionic antimicrobial agents such as triclosan. Certain flavour oils, such as thymol, may also have antimicrobial activity. Such agents are disclosed in U.S. Pat. No. 2,946,725, Jul. 26, 1960, to Norris et al. and U.S. Pat. No. 4,051,234, Sep. 27, 1977 to Gieske et al. Also useful is sodium chlorite, described in WO 99/43290, incorporated herein by reference.

Antimicrobial agents, if present, are typically included at levels of from about 0.01% to about 10%. It is preferred to keep the level of stannous and cationic antimicrobial agents to less than 5%, preferably less than 1% to avoid staining problems.

Preferred antimicrobial agents are non-cationic antimicrobial agent, such as those described in U.S. Pat. No. 5,037,637. A particularly preferred antimicrobial agent is 2′,4,4′-trichloro-2-hydroxy-diphenyl ether (triclosan).

Thickeners

Organic thickeners suitable for the present invention include hydroxypropyl methylcellulose, hydroxyethyl cellulose, sodium carboxymethyl cellulose, xanthan gum, guar gum, tragacanth gum, karaya gum, arabic gum, Irish moss, starch, alginate and carrageenan. Most preferred are the polysaccharide gums, especially xanthan, guar and carrageenan. Amounts of organic thickeners may range from 0.1 to 3%, preferably from 0.55 to 1.5%, optimally from 0.7 to 1.5% by weight. To avoid excessive thickening of the dentifrice it preferably comprises less than about 2% of polymeric thickeners, which are additional to any polyoxyethylene that may be used.

Silicone oils

An optional ingredient in the present compositions is a silicone oil. Silicone oils can be useful as plaque barriers, as disclosed in WO 96/19191, incorporated herein by reference. Suitable classes of silicone oils include, but are not limited to, dimethicones, dimethiconols, dimethicone copolyols and aminoalkylsilicones, preferred silicone oils are selected from dimethicone copolyols and aminoalkylsilicones, more preferably from dimethicone copolyols. Preferred dimethicone copolyols are selected from C₁₂ to C₂₀ alkyl dimethicone copolyols and mixtures thereof. Highly preferred is cetyl dimethicone copolyol marketed under the trade name Abil EM90. The silicone oil is useful as an antipiaque agent and helps to prevent re-soiling of teeth. It is generally used at a level of from about 0.1% to about 15%, preferably from about 0.5% to about 5%, more preferably from about 0.5% to about 3% by weight.

Other adjuvants

Sweetening agents such as sodium saccharin, sodium cyclamate, Acesulfame K, aspartame, sucrose and the like may be included at levels from about 0.1 to 5% by weight. Other additives may also be incorporated including preservatives, opacifiers and colorants. Typical colorants are D&C Yellow No. 10, FD&C Blue No. 1, FD&C Red No. 40, D&C Red No. 33 and combinations thereof. Levels of the colorant may range from 0.0001 to 0.1, preferably from 0.001 to 0.01% by weight.

Use

The liquid dentifrice of this invention can be used in a wholly conventional manner. It is specifically designed, however, for use with a toothbrush having a small head, in particular an electric toothbrush. In a further aspect, the invention relates to a method of cleaning teeth comprising dosing less than 1 ml of a liquid dentifrice, as herein described, onto or into a cleaning implement and cleaning the teeth with the dosed implement. The cleaning implement can be, for example, a sponge or a brush, preferably it is a toothbrush, and more particularly an electric toothbrush, especially one whose brush head has a surface area, measured on the working surface, of less than about 1.5 cm². In preferred embodiments the toothbrush is a sonic toothbrush.

The dentifrice herein is particularly suited to a dentifrice dispensing toothbrush comprising a brush portion and an integrated dentifrice reservoir, which reservoir is able to contain a liquid dentifrice as herein described, wherein the toothbrush is adapted to dispense a unit dose of less than about 1 ml of the dentifrice. A suitable housing, cartridge and applicator assembly for such a toothbrush is described in WO 02/41802 which is incorporated herein by reference in its entirety. The dose control can be by an electronically controlled pump or, for example, by a resilient push button operated mechanical pump placed over the reservoir, or in line with a conduit leading from it, such that a single manual depression of the button displaces less than about 1 ml of the dentifrice from the reservoir.

In yet a further aspect, the invention relates to a liquid dentifrice as described herein, wherein the package has a dispensing orifice of cross-sectional area of less than about 10 mm². The packaged dentifrice is optionally combined with a set of instructions for dispensing onto and use with an electric toothbrush. The packaged dentifrice can be a replaceable reservoir for use with an electric toothbrush having an integrated dentifrice reservoir and the optional instructions may comprise directions for replacing the reservoir in the toothbrush. For this application in particular, the dentifrice is suited for dispensing through narrow dispensing outlets such as a conduit leading from the reservoir to the brush head. In this preferred embodiment, wherein such a conduit is considered to be part of the dispensing outlet of the package, the dispensing orifice preferably has a cross-sectional area of less than about 10 mm², more preferably less than about 5 mm2.

### EXAMPLES

The following examples will more fully illustrate embodiments of this invention.

| | Example # | | | |
|---|---|---|---|---|
| | I | II | III | IV |
| Material Name | Wt % | Wt % | Wt % | Wt % |
| Sorbitol (70%) | 43.2 | 41.9 | 41.9 | 52.0 |
| Glycerin | 10.0 | 10.0 | 10.0 | 10.0 |
| Sodium fluoride | 0.32 | 0.32 | 0.32 | 0.32 |
| Xanthan gum | 0.5 | 1.0 | 1.0 | 1.0 |
| Sodium alkyl sulphate, 28% | 9.0 | 9.0 | 9.0 | 9.0 |
| Precipitated silica | — | 3.0 | 10.0 | 5.0 |
| PEG-6 | 5.0 | 5.0 | 5.0 | — |
| PEG-12 | — | — | — | 3.0 |
| PEG-20 M | 2.0 | — | 1.3 | 4.0 |
| Triclosan | — | 0.3 | — | — |
| Sodium saccharin | 0.45 | 0.45 | 0.45 | 0.45 |
| Flavour | 1.6 | 1.7 | 1.9 | 1.7 |
| Preservative | 0.1 | 0.1 | 0.1 | 0.1 |
| CI 42090 FD&C Blue No. 1 | 0.002 | 0.002 | 0.002 | 0.002 |
| Water | to 100% | to 100% | to 100% | to 100% |

The liquid dentifrice of Examples I-V can be packaged in a simple tube or in a reservoir adapted for use with a toothbrush having an integrated dentifrice reservoir. On dispensing and use of less than 1 ml of the dentifrice, satisfactory foaming, cleaning and flavour impact is obtained.

The foregoing description and Examples illustrate selected embodiments of the present invention. While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A liquid dentifrice having a viscosity of less than about 500 Pa.s at 0.1 s-1 at 25° C. and comprising:
a) greater than about 2% by weight of a surfactant;
b) greater than about 1.2% flavour; and
c) greater than 50% liquid carrier materials, comprising water and humectants in a ratio of from about 0.65 to 1.5.

2. A liquid dentifrice having a viscosity of less than about 500 Pa.s at 0.1 s⁻¹ at 25° C. and comprising:
a) at least about 0.1% of a polyoxethylene having a molecular weight greater than 200,000;
b) at least about 2% of a dental abrasive; and
c) greater than 50% liquid carrier materials.

3. A liquid dentifrice according to claim 1 comprising at least 0.5% of a polyoxethylene having a molecular weight greater than about 200,000.

4. A liquid dentifrice according to claim 3 comprising less than 2% of additional polymeric thickeners.

5. A liquid dentifrice according to claim 1 comprising from about 2% to about 20% dental abrasive.

6. A liquid dentifrice according to claim 1 which comprises at least about 0.5% anionic surfactant.

7. A liquid dentifrice according to claim 6 which comprises at least about 2% anionic surfactant.

8. A liquid dentifrice according to claim 1 which comprises from about 30% to about 50% total water.

9. The liquid dentifrice according to claim 1 which is contained in a package having a dispensing orifice of cross-sectional area of less than about 10 mm².

10. The liquid dentifrice according to claim 9 wherein the package is printed with instructions for use of the liquid dentifrice with an electric toothbrush.

11. A method of cleaning teeth according to claim 9 comprising dosing less than 1 ml of the liquid dentifrice according to claim 1 onto the head of an electric toothbrush and cleaning the teeth with the dosed toothbrush head.

12. The method of claim 11 wherein the electric toothbrush is a sonic toothbrush.

13. A dentifrice dispensing toothbrush comprising a brush portion and an integrated dentifrice reservoir, the reservoir comprising a liquid dentifrice according to claim 1 wherein the toothbrush is adapted to dispense a unit dose of less than 1 ml of the dentifrice.

14. A liquid dentifrice according to claim 2 comprising at least 0.5% of a polyoxethylene having a molecular weight greater than about 200,000.

15. A liquid dentifrice according to claim 14 comprising less than 2% of additional polymeric thickeners.

16. A liquid dentifrice according to claim 2 comprising from about 2% to about 20% dental abrasive.

17. A liquid dentifrice according to claim 2 which comprises at least about 0.5% anionic surfactant.

18. A liquid dentifrice according to claim 17 which comprises at least about 2% anionic surfactant.

19. A liquid dentifrice according to claim 2 which comprises from about 30% to about 50% total water.

20. The liquid dentifrice according to claim 2 which is contained in a package having a dispensing orifice of cross-sectional area of less than about 10 mm².

21. The liquid dentifrice according to claim 20 wherein the package is printed with instructions for use of the liquid dentifrice with an electric toothbrush.

22. A method of cleaning teeth according to claim 20 comprising dosing less than 1 ml of the liquid dentifrice according to claim 2 onto the head of an electric toothbrush and cleaning the teeth with the dosed toothbrush head.

23. The method of claim 22 wherein the electric toothbrush is a sonic toothbrush.

24. A dentifrice dispensing toothbrush comprising a brush portion and an integrated dentifrice reservoir, the reservoir comprising a liquid dentifrice according to claim 2 wherein the toothbrush is adapted to dispense a unit dose of less than 1 ml of the dentifrice.
